(19) United States
(12) Patent Application Publication
(10) Pub. No.: US 2014/0364770 A1
(43) Pub. Date: Dec. 11, 2014

(54) ACCELEROMETER-BASED SLEEP ANALYSIS

(71) Applicant: MOTOROLA MOBILITY LLC, Libertyville, IL (US)

(72) Inventors: Andrew M. Slonneger, Crystal Lake, IL (US); Sajid I. Dalvi, Aurora, IL (US); Viditha Hanumanthareddy, Buffalo Grove, IL (US)

(21) Appl. No.: 13/939,203

(22) Filed: Jul. 11, 2013

Related U.S. Application Data
(60) Provisional application No. 61/831,638, filed on Jun. 6, 2013.

(51) Int. Cl. A61B 5/00; G01P 21/00

(52) U.S. Cl. CPC A61B 5/4812; G01P 21/00
USPC 600/595; 702/104

(57) ABSTRACT

Disclosed are systems and methods for sleep analysis using an accelerometer-based user device. In one aspect, motion-based data from an accelerometer associated with the user device are used to determine one or more average normalized activity levels based on a series of consecutive motion-based data samples. An epoch decision value is determined using one or more consecutive normalized activity levels, and a sleep-decision window value is determined using a series of one or more consecutive epoch decision values. A sleep-state value is determined using the epoch values from the series of epoch values and is compared with reference sleep-state values to determine the sleep state of the user of the device. In some examples, the systems and methods may include a method for dynamically calibrating the data received from the accelerometer.

### CROSS-REFERENCE TO RELATED APPLICATIONS

The present application claims priority to U.S. Provisional Patent Application 61/831,638, filed on Jun. 6, 2013, which is incorporated herein by reference in its entirety.

### TECHNICAL FIELD

The present disclosure relates to sleep analysis, and more particularly, to accelerometer-based methods for sleep analysis.

### BACKGROUND

Within the medical and fitness fields, sleep-analysis testing is performed to gauge the timing of, duration of, and perceived quality of sleep. Such sleep-analysis data can be used by professionals or individuals to help prevent or combat sleep deprivation. Sleep deprivation is believed be associated with a greater susceptibility to physical illness, greater occurrence of emotional and mental health problems, negative mood swings, and diminished capacity to concentrate, remember, learn and complete tasks. In light of the deleterious consequences of sleep deprivation, systems, methods, and apparatus for monitoring the timing, duration, and quality of sleep have been developed.

However, most currently used methods for monitoring and analyzing sleep conditions and sleep states are invasive to the user or lack accuracy. Many widely used sleep-analysis methods use polysomnography (“PSG”) to monitor sleep states or to detect various sleep disorders. PSG is a medical procedure that generally involves a full night of testing in a sleep laboratory to monitor temporal variations in the amplitude of the user's sleep-impacted physiological parameters, including, for example, blood-oxygen saturation, heart rate, upper-respiratory airflow, thorax and abdomen respiration efforts, electrical activity of the brain, electrical activity related to movement of the eyes, and electrical activity related to muscle movement. PSG testing procedures have been cost prohibitive for many patients as they require administration by a professional in a laboratory setting. While recent limited PSG monitoring procedures may be used in a patient's home, such procedures nonetheless require administration by a technician.

In an attempt to avoid the disadvantages of PSG and simulated PSG sleep-analysis methods, methods for sleep analysis have been developed using motion-related data associated with a sleeping user's arm movements. The arm movements are sensed and associated with various states of sleep. Accelerometer-based devices used to monitor sleep states without professional administration typically involve the use of a motion sensor (i.e., an accelerometer) to quantify the temporal variation in a user's motion at the sensor's location. The motions are then analyzed to compute a sleep score based on the magnitude of movements recorded during the monitoring period.

This Background section represents the observations of the inventors, which are provided as a guide to the reader in attempting to search for and identify any prior art of interest. Nothing in this Background section is intended to represent, nor to fully describe, such prior art.

### BRIEF DESCRIPTION OF THE SEVERAL VIEWS OF THE DRAWINGS

While the appended claims set forth the features of the present techniques with particularity, these techniques, together with their objects and advantages, may be best understood from the following detailed description taken in conjunction with the accompanying drawings of which:

FIG. 1 is a generalized schematic of an example device within which the presently disclosed innovations may be implemented;

FIG. 2 is a generalized schematic of a system within which the presently disclosed innovations may be implemented;

FIG. 3 is a schematic diagram with respect to which an accelerometer of the presently disclosed innovations may be configured;

FIG. 4 illustrates an example system for performing sleep analysis based on the presently disclosed innovations;

FIG. 5 illustrates an example system for performing dynamic calibration of the accelerometer of the presently disclosed innovations;

FIG. 6 illustrates an example system for determining a sleep state for a given period using the accelerometer data of the presently disclosed innovations;

FIG. 7 is a flowchart illustrating a process for determining if, for a given state, an accelerometer can be dynamically calibrated and for setting minimum and maximum values for given parameters;

FIG. 8 shows a continuation of the flowchart of FIG. 7, wherein the accelerometer is dynamically calibrated;

FIG. 9 is a flowchart illustrating a process of applying dynamic calibration to raw accelerometer data; and

FIG. 10 is a flowchart illustrating a method for determining a sleep state based on accelerometer data.

### DETAILED DESCRIPTION

Turning to the drawings, wherein like reference numerals refer to like elements, techniques of the present disclosure are illustrated as being implemented in a suitable environment. The following description is based on embodiments of the claims and should not be taken as limiting the claims with regard to alternative embodiments that are not explicitly described herein.

The inventors have observed that it is desirable to improve upon existing accelerometer-based sleep-analysis methods to provide more accurate readings and to provide more detailed analysis of sleep states. Presently disclosed systems and methods may improve upon accuracy of readings from the accelerometer and may present data in a comprehensive, time-based manner (i.e., a graph showing sleep state vs. time). Sleep may be monitored using methods or associated devices employing motion-related data captured by an accelerometer associated with the user.

In some disclosed sleep-analysis methods, the methods discern between three sleep states: deep sleep, light sleep, and awake. Data indicating such states, based on predetermined metrics, are processed to ascertain health of a sleep cycle, sleep quality, or other sleep-related conditions or measurements. In keeping with this aspect of the disclosure, the metrics used to determine a sleep state of the user include, for example, the Metabolic Equivalent of Task parameter.

Some example user devices for performing accelerometer-based sleep analysis implement, for example, a stability threshold function to turn off the accelerometer when it is stable. Such stability-threshold functions may reduce current drain in the user device.

Sleep-analysis methods that involve the use of an accelerometer-based user device are often prone to error. In particular, sleep analysis using an accelerometer-based user device can involve sensitive methods of implementation with respect to calibration errors associated with the accelerometer. Further, additive errors (i.e., offsets) or multiplicative errors (i.e., scaling errors) may exist. Methods to reduce such errors are discussed with respect to various embodiments.

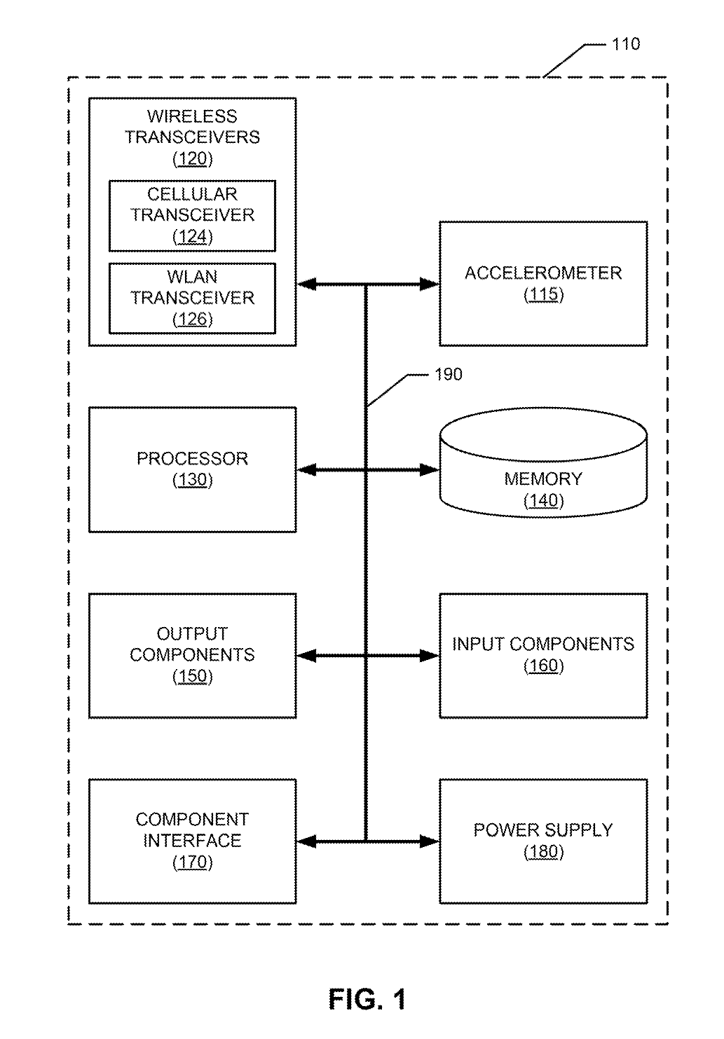

Turning now to a more detailed description in view of the attached figures, the schematic diagram of FIG. 1 shows an exemplary device within which aspects of the present disclosure may be implemented. In particular, the schematic diagram illustrates a user device 110 including exemplary internal components. Such a user device may be associated with the user in the form of a wrist-watch; however, the device is not limited to the form of a wrist watch. Internal components of the user device 110 may include wireless transceivers 120, a processor 130, a memory 140, one or more output components 150, one or more input components 160, an accelerometer 115, as well as one or more additional optional sensors.

The processor 130 can be any of a microprocessor, microcomputer, application-specific integrated circuit, or the like. For example, the processor 130 can be implemented by one or more microprocessors or controllers from any desired family or manufacturer. Similarly, the memory 140 may reside on the same integrated circuit as the processor 130. Additionally or alternatively, the memory 140 may be accessed via a network, e.g., via cloud-based storage. The memory 140 may include a random-access memory (i.e., Synchronous Dynamic Random-Access Memory, Dynamic Random-Access Memory, RAMBUS Dynamic Random-Access Memory, or any other type of random-access memory device). Additionally or alternatively, the memory 140 may include a read-only memory (i.e., a hard drive, flash memory, or any other desired type of memory device).

The data that are stored by the memory 140 can include one or more operating systems or applications as well as informational data. Each operating system is implemented via executable instructions stored in a non-transitory computer-readable medium to control basic functions of the electronic device 110. Such functions may include, for example, interaction among various internal components, communication with external devices via the wireless transceivers 120 or via the component interface 170, and storage and retrieval of applications and data to and from the memory 140.

The device 110 may also include a component interface 170 to provide a direct connection to auxiliary components or to accessories. The device 110 may include a power supply 180, such as a battery, for providing power to the device components. In an embodiment, all or some of the internal components communicate with one another by way of one or more internal communication links 190, such as an internal bus.

With respect to programs, sometimes also referred to as applications, each program is implemented via computer-executable code, e.g., read from the above non-transitory medium or otherwise, that utilizes the operating system to provide more specific functionality, such as file-system service and handling of protected and unprotected data stored in the memory 140. Although many such programs govern standard or required functionality of the user device 110, in many cases the programs include applications governing optional or specialized functionality, which can be provided, in some cases, by third-party vendors unrelated to the device manufacturer.

Finally, with respect to informational data, this non-executable information can be referenced, manipulated, or written by an operating system or program. Such informational data can include, for example, data that are preprogrammed into the device 110 during manufacture, data that are created by the device 110, or any of a variety of types of information that are uploaded to, downloaded from, or otherwise accessed at servers or other devices with which the device 110 is in communication during its ongoing operation.

In an embodiment, the device 110 can be programmed such that the processor 130 and the memory 140 interact with the other components of the device 110 to perform a variety of functions. The processor 130 may include or implement various modules and execute programs for initiating different activities such as launching an application, transferring data, and toggling through various graphical user interface objects (e.g., toggling through various icons that are linked to executable applications).

The wireless transceivers 120 can include, for example, both a cellular transceiver 124 and a wireless local area network (“WLAN”) transceiver 126. Each of the wireless transceivers 120 utilizes a wireless technology for communication, such as cellular-based communication technologies including analog communications, digital communications, next-generation communications, variants thereof, or peer-to-peer or ad hoc communication technologies such as Bluetooth or other wireless communication technologies.

Exemplary operation of the wireless transceivers 120 in conjunction with other internal components of the device 110 can take a variety of forms and can include, for example, operation in which, upon receipt of wireless signals, the internal components detect communication signals, and one of the transceivers 120 demodulates the communication signals to recover incoming information, such as voice or data, transmitted by the wireless signals. After receiving the incoming information from a transceiver 120, the processor 130 formats the incoming information for the one or more output components 150. Likewise, for transmission of wireless signals, the processor 130 formats outgoing information and conveys the outgoing information to one or more of the wireless transceivers 120 for modulation as communication signals. The wireless transceivers 120 convey the modulated signals to a remote device, such as a cell tower or an access point (not shown).

The accelerometer 115 measures acceleration along coordinate axes (i.e., acceleration along the x, y, and z axes). The measured acceleration is used to determine motion of the user device 110, which may then be used to calculate a level of the user's motion. Accelerometer 115 may read acceleration associated with the phenomenon of weight experienced by the user device 110 in the frame of reference of the accelerometer 115. The accelerometer 115 may measure g-force acceleration, which is measured as force-per-unit mass. Because g-force is perceived as a weight, any g-force can be described as a “weight-per-unit mass.”

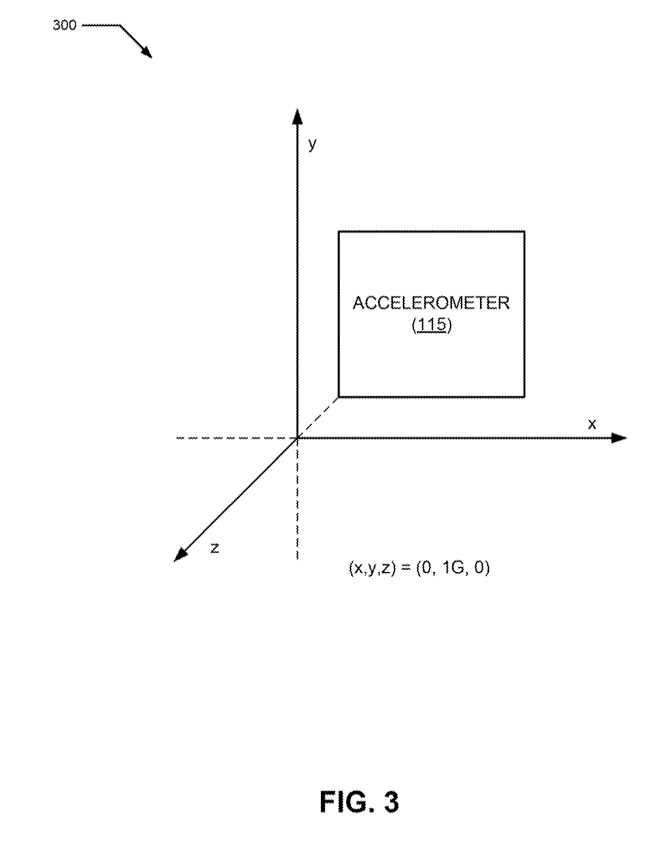

The accelerometer 115 may read values in one, two, or three dimensions (i.e., at least one or more of the coordinate axes: x, y, and z). In the proceeding examples, the accelerometer 115 reads acceleration in all three dimensions: x, y, and z. Device alignment of the accelerometer is shown in FIG. 3 for a three-dimensional (“3-D”) Cartesian coordinate system. In particular, for the device position shown in FIG. 3, the x-axis is horizontal (left to right) and has no gravity component, the y-axis is vertical (bottom to top) and has the entire gravity component, and the z-axis is horizontal (back to front) and has no gravity component. As seen in FIG. 3, the accelerometer 115, at rest, nominally reads directional values of:

(x,y,x)=(0,1G,0)

Thus, the acceleration, at rest, of the accelerometer 115 is the force of gravity, or “1G” in terms of g-force, as defined as the magnitude of the 3-D acceleration vector:

mag=|x,y,z|=\sqrt{x²+y²+z²}

Therefore, when the accelerometer is at rest:

mag=1G

Readings of the acceleration vector or magnitude of the acceleration vector are passed to other elements within the user device 110 for further analysis.

Ideally, a stationary user device 110 containing an accelerometer 115 will show no difference between consecutive acceleration values for a given axis when the device is stationary (ideal stationary acceleration being 1G due to gravity). However, there may be noise associated with the accelerometer 115; thus, consecutive acceleration values at a stationary state may not be identical.

The user device 110 may also include other sensors (not shown) including, but not limited to, proximity sensors, gyroscopes, haptic sensors, light sensors, temperature sensors, biological sensors, chemical sensors, humidity sensors, or any other type of sensor for providing pertinent information related to sleep analysis.

Actions that actuate one or more input components 160 can include, for example, powering on, opening, unlocking, moving, or operating the device 110. For example, upon power on, a “home screen” with a predetermined set of application icons may be displayed on a touch screen of the device 110.

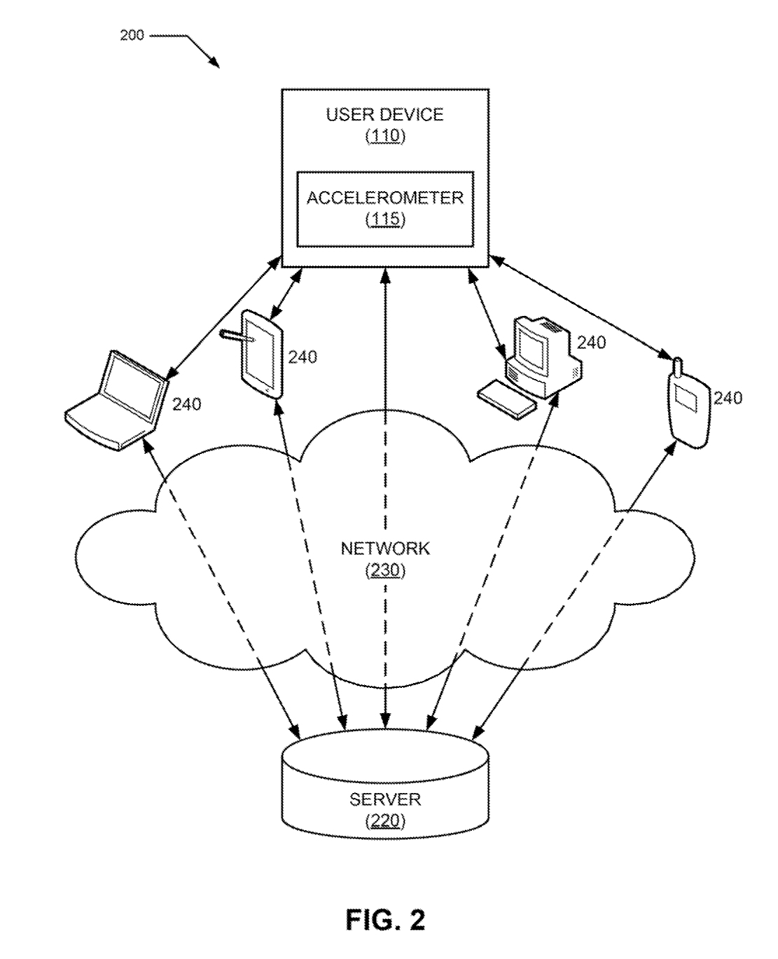

While the accelerometer-based user device 110 may perform sleep-analysis methods internally and display results via an included screen, in other examples, the sleep-analysis system communicates the sleep-analysis data externally as seen in the system 200 of FIG. 2.

In this system 200, the user device 110 transmits accelerometer data or processed sleep-analysis data to a server 220 via a network 230. Such data may be transferred from the user device 110 using the wireless transceivers 120, which can include, as shown in FIG. 1, a cellular transceiver 124 or a WLAN transceiver 126. The network 230 may be any suitable network for transmitting data from the user device 110 to the server 220. The network 230 can include, for example, one or more of the Internet, a wide-area network, a local-area network, a personal area network (“PAN”), a Bluetooth link, or any other suitable communicative network or link over which electronic data may be transferred.

The server 220 may be communicatively coupled with one or more computing devices 240, such as a desktop computer, a laptop computer, a tablet computing device, a cellular telephone, a smart phone, a smart watch, a personal digital assistant, a set-top box, or any other computing device which may process or display data. The computing devices 240 may receive data related to sleep analysis from the server 220, and may process sleep-analysis data and report processed data back to the server 220; this processed data may be transmitted from the server 220 to any other computing devices 240 or to the user device 110 for display or further processing.

Additionally or alternatively, the user device 110 is directly connected via hardwired means (i.e., a USB cable or any other suitable data-transfer hardwiring) or network means (i.e., over WiFi, a Bluetooth connection, a PAN, etc.) to the one or more computing devices 240. The computing devices 240 may process or display sleep-analysis data received directly from the user device 110.

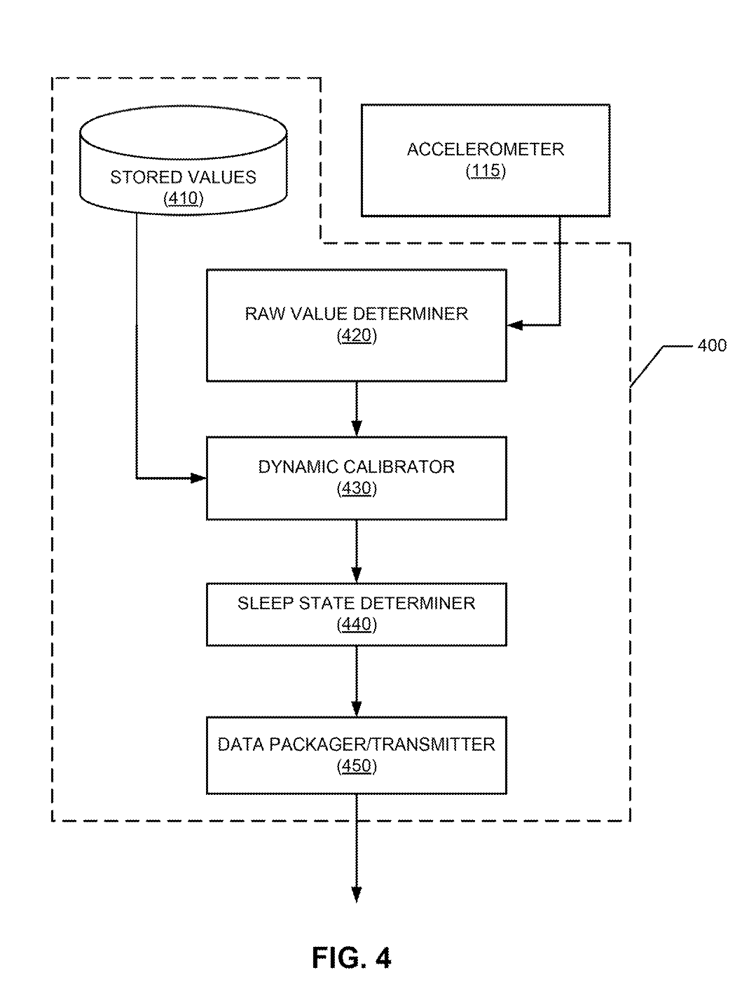

Turning to FIG. 4, the sleep-analysis system 400 is implemented in keeping with instructions stored on the memory 140 and executed by the processor 130. The system 400 receives data from the accelerometer 115 as well as stored values 410 and determines raw values using a raw-value determiner 420. The sleep-analysis system 400 then employs an offset corrector 430 to correct errors detected in the data produced by the accelerometer 115. The sleep-analysis system 400 then determines sleep-state data based on the corrected data received, using a sleep-state determiner 440. Sleep-state data can be compiled or packaged for transmission by a data packager/transmitter 450. The sleep-analysis system 400 then passes the packaged or compiled data to a downstream source.

The actual acceleration values received from the accelerometer 115 by the raw-value determiner 420 are represented in the form:

(x,y,z)

However, the actual acceleration values may be skewed by a scaling error (denoted “s”) or an offset error (denoted “e”), with s and e defined as:

s=(sₓ,s_{y},s_{z}) e=(eₓ,e_{y},e_{z})

Using these values of s and e, the raw values of the output of the accelerometer (\ddot{x},ÿ,\ddot{z}); can be defined as:

$$( \hat{x} , \hat{y} , \hat{z} ) = ( \frac{x}{{s}_{x}} - \text{?} , \frac{y}{{s}_{y}} - \text{?} , \frac{z}{{s}_{z}} - \text{?} )$$

$$\text{?} \text{indicates text missing or illegible when filed}$$

This raw output may then be processed by the dynamic calibrator 430.

The dynamic calibrator 430 may estimate values for a scaling-error correction or offset error correction and apply these corrections to the raw values of the output of the accelerometer (\ddot{x},ÿ,\ddot{z}) to obtain a better estimate of the actual acceleration. The dynamic calibration method performed by the dynamic calibrator 430 may include incrementally estimating offset correction (incrementally estimating additive errors), determining offset correction (estimating additive errors), determining scaling correction (estimating multiplicative errors), applying offset correction (compensating for additive errors), or applying scaling correction (compensating for multiplicative errors). The initial values stored (for example, the stored values 410) for the scaling-error correction and offset-error correction represent a state in which no error correction is needed, that is:

s=(1,1,1) e=(0,0,0)

Using such initial values allows application of offset and scaling correction to always occur. Additionally, a flag may be set once any of the six values changes, with corrective action conditioned on the set flag. Similarly, a flag per axis may be used with correction application per axis depending on each axis' respective flag.

In an embodiment, the dynamic calibrator 430 applies the scaling-error correction and offset-error correction as follows:

(x,y,z)=(sₓ(\hat{x}+eₓ),s_{y}(ŷ+e_{y}),s_{z}(\hat{z}+e_{z})

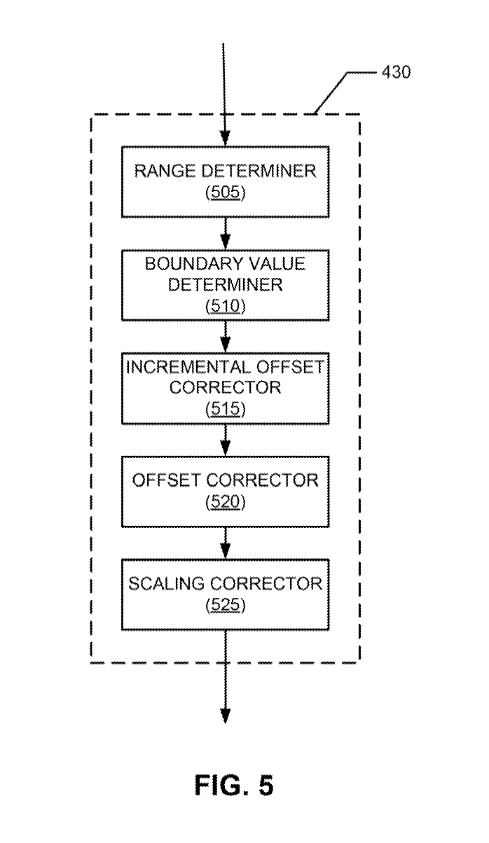

The accelerometer output processed by the dynamic calibrator 430 may then be read as input by the sleep-state determiner 440. An example dynamic calibrator 430 is described in greater detail with reference to FIG. 5.

The accelerometer-sensor data used by the sleep-state determiner 440 may be the estimated acceleration of the accelerometer 115 output by the dynamic calibrator 430. The sleep-state determiner 440 uses the accelerometer data or user-input data to determine decision values for pre-determined time intervals. The decision values are used by the sleep-state determiner 440 to determine the sleep state of the user. Sleep states recognized by the sleep-state determiner 440 from the decision values include, but are not necessarily limited to, deep sleep, light sleep, and awake.

The sleep-state determiner 440 determines an average normalized activity level over one second using a number of accelerator-magnitude samples (N samples). The average normalized activity level is used to determine a sample measure over one second for the accelerometer 115. Consecutive sample measures, each taken over a one second interval, may be used to determine a sample activity count using set high and low thresholds. 60 consecutive sample activity counts may be used to determine an epoch activity count, the epoch activity count being a sample metric for a one-minute epoch. The epoch activity rate may be compared to thresholds for sleep states to determine a sleep-state epoch decision.

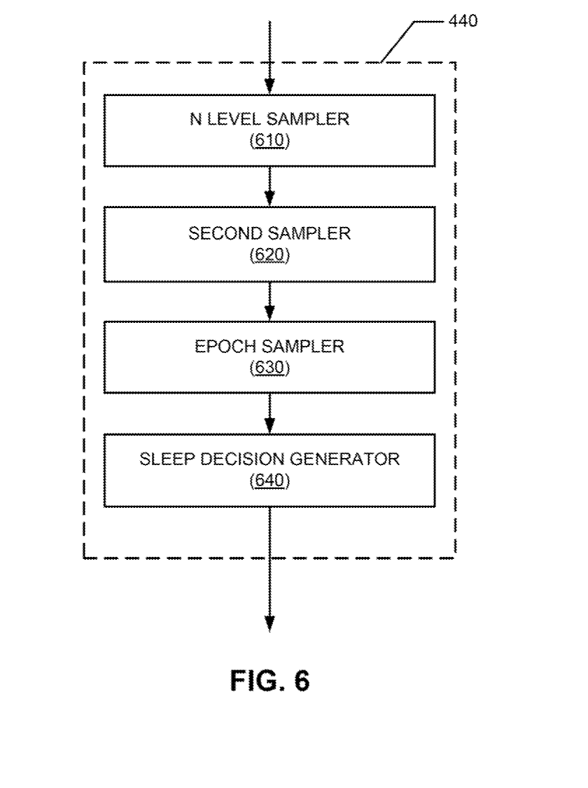

A series of epoch decisions is used to determine a sleep decision, the sleep decision being the sleep state of the user during a sleep-decision window. For example, a sleep-decision window of five minutes may combine five consecutive one-minute epoch decisions. In an embodiment, in order to determine the sleep state, the sleep-state determiner 440 chooses the maximum value from the consecutive epochs within the sleep-state window. An example sleep-state determiner 440 is described in greater detail with reference to FIG. 6.

The data packager/transmitter 450 packages data for use by elements internal to the user device 110 (i.e., the memory 140, the output components 150, the component interface 170, the wireless transceivers 120, etc.) or external devices (i.e., the server 220, the computing devices 240, etc.). For example, the data may be compiled into a chart or graph showing sleep states at each sleep-decision window over a given period of time for display to the user via an output component 150 of the user device (i.e., a visual display), or the data may be stored on the server 220 or memory 140 for later viewing on the user device 110 or on another computing device 240.

FIG. 5 is a schematic illustration of an example dynamic calibrator 430 which can be used in association with the system 400 of FIG. 4. The dynamic calibrator 430 may include a range determiner 505, a boundary-value determiner 510, an incremental-offset determiner 515, an offset corrector 520, and a scaling corrector 525.

In an example embodiment, correction values are only updated when the user device 110 is stationary. Further, dynamic calibration methods may determine errors only when the current accelerometer magnitude is within a correctable range with respect to 1G. For example, a correctable range may be 14% of 1G. Thus, the range determiner 505 determines whether or not dynamic calibration should be performed based on the current accelerometer magnitude (“magraw”) such that:

$$if \frac{\vert {mag}_{raw} - {mag}_{ideal} \vert}{{mag}_{ideal}} = \frac{\vert {mag}_{raw} - 1 G \vert}{1 G} < 14 \%$$

$$where \text{:}$$

$${mag}_{raw} = \vert \hat{x} , \hat{y} , \hat{z} \vert = \sqrt{{\hat{x}}^{2} + {\hat{y}}^{2} + {\hat{z}}^{2}}$$

$${mag}_{ideal} = \vert x , y , z \vert = \sqrt{{x}^{2} + {y}^{2} + {z}^{2}} = 1 G$$

If the value for the current accelerometer magnitude is within a correctable range of 1 G, as determined by the range determiner 505, then dynamic calibration may be performed.

The boundary value determiner 510 determines and records a minimum and maximum value for each axis (x, y, z) and updates said minimum and maximum values based on the current raw accelerometer output, e.g., via the following conditions:

if (\hat{x}>x_{max□}), then xₘₐₓ=\hat{x}

if (ŷ>y_{max□}), then yₘₐₓ=ŷ

if (\hat{z}>z_{max□}), then zₘₐₓ=\hat{z}

if (\hat{x}>x_{min□}), then xₘᵢₙ=\hat{x}

if (ŷ>y_{min□}), then yₘᵢₙ=ŷ

if (\hat{z}>z_{min□}), then zₘᵢₙ=\hat{z}

In some examples, the ideal difference between maximum value and minimum value for each axis is 2G, that is:

xₘₐₓ−xₘᵢₙ−2G

yₘₐₓ−yₘᵢₙ=2G

zₘₐₓ−zₘᵢₙ=2G

In some examples, the steps of calculating the offset correction and scaling correction for an axis occur when the minimum and maximum values are separated by nearly 2G. For example, offset correction and scaling correction for x may occur when the minimum value and maximum value are separated by more than 92% of 2G, that is:

$$\frac{{\hat{x}}_{max} - {\hat{x}}_{min}}{2 G} > 92 \%$$

However, in an embodiment, incremental estimates of the offset correction occur prior to obtaining this minimum separation of the minimum and maximum values.

When the recorded minimum and maximum values for an axis do not yet have a large enough separation to determine the offset-correction and scaling-correction values, then the error estimates may be incrementally calculated using the incremental offset corrector 515. In an embodiment wherein the magnitude of the corrected value is too large, the offset may be incrementally adjusted as well. For example:

then, if (\hat{x}>0), sₓ=eₓ−0.8%(1G)

then, if (\hat{x}<0), sₓ=eₓ−0.8%(1G)

When the magnitude of the corrected value is too small and the corrected values of the other two axes have small magnitudes, the offset may be adjusted. For example:

$$and \frac{\hat{y} + \text{?}}{1 G} < 13 \%$$

$$and \frac{\hat{z} + \text{?}}{1 G} < 15 \%$$

$$\text{?} \text{indicates text missing or illegible when filed}$$

The incremental offset corrector 515 may repeat this operation until the recorded minimum and maximum values for an axis are far enough apart, for example, when the minimum and maximum values are nearly 2G apart (i.e., 92%).

If the recorded minimum and maximum value for an example are far enough apart, e.g., if:

$$\frac{{\hat{x}}_{max} - {\hat{x}}_{min}}{2 G} > 92 \% ,$$

and if the mean of the minimum value and the maximum value is within a correctable range of 0, e.g., if:

$$\text{?} \text{?} max + \text{?} min \text{?} / 16 < 16 \%$$

$$\text{?} \text{indicates text missing or illegible when filed}$$

then the offset corrector 520 determines the offset correction (“ex”) as follows, in an embodiment:

$$\text{?} if ( {\hat{x}}_{max} + {\hat{x}}_{min} • ) < 0 , \\ {e}_{x} = \frac{{\hat{x}}_{max} + {\hat{x}}_{min}}{2}$$

$$\text{?} \text{indicates text missing or illegible when filed}$$

while the offset corrector 520 may have the following constraint:

$$where \vert \text{?} \vert < \text{?} \% ( \text{?} )$$

$$\text{?} \text{indicates text missing or illegible when filed}$$

Further, using the same conditions for minimum and maximum values and correctible range of the axis value, the scaling corrector 525 can determine the scaling correction (“sx”) as follows (which may be subject to a max deviation from 1.0):

$${s}_{x} = \frac{2 G}{{R}_{max} - {R}_{min} •} where$$

$$\vert {s}_{x} - 1 \vert < 10 \%$$

Once the offset correction and scaling correction are determined, the offset correction may be applied to the raw x value by the offset corrector 520. Similarly, the scaling correction may be applied to the raw value for x by the scaling corrector 525. The preceding determinations are repeated sequentially or in parallel for all three axes (x, y, z).

Three-dimensional acceleration data (x, y, z) are received by an exemplary sleep-state determiner as seen in FIG. 6. The 3-D acceleration data may be read as, or converted to, a magnitude value (“mag”) defined as:

mag=|x,y,z|=\sqrt{x²+y²+z²}

The N level sampler 610 may receive mag values at a rate of N samples per second. Using the N samples, the second sampler 620 then determines an average normalized activity level (“alk”) over one second for accelerometer samples taken at the rate N Hertz:

$${al}_{k} = \frac{1}{1 G} \frac{1}{N} \text{?} \text{?}$$

$$\text{?} \text{indicates text missing or illegible when filed}$$

Using the average normalized activity level, the second sampler 620 determines a sample measure (“smk”) for the sampled second. For example, the sample measure may be defined as:

smₖ=19.2(alₖ)−19.4

Using the sample measure value and predetermined high and low threshold values for the sample measure (“Tsm-high,” “Tsm-low”), the second sampler 620 determines a sample activity count (“sac”). The sample activity count for each second compares the sample metric to thresholds (Tsm-high, Tsm-low) to determine activity counts associated with a sleep state. For example, using predetermined threshold values and activity count values:

$${T}_{sm - high} = 8$$

$${T}_{sm - low} = 1.5$$

$${sac}_{k} = \{ \begin{matrix}15 & {sm}_{k} > {T}_{sm - high} \\ 1 & {T}_{sm - low} < {sm}_{k} \leq {T}_{sm - high} \\ 0 & {sm}_{k} \leq {T}_{sm - low}\end{matrix} .$$

Using determined sample activity counts (“sack”), the epoch sampler 630 determines a sleep state during a one-minute epoch. The epoch sampler 630 then determines an epoch activity count (“eac”) for each minute. The metric for an epoch activity count may be based on 60 activity count samples (M=60) per minute, e.g.:

$${eac}_{m} = \text{?} {sac}_{k} . \\ \text{?} \text{indicates text missing or illegible when filed}$$

The epoch activity counts may then be used to determine an epoch measure, which defines a sleep state for the analyzed one-minute epoch. The epoch activity count applies predetermined threshold values signifying a sleep state of awake (“Tem-awake”) and of deep sleep (“Tem-deep”). For example, the threshold values may be defined as:

T_{em-awake}=61

T_{em-deep}=5.

Using the thresholds and the epoch activity count samples, the epoch sampler 630 determines an epoch measure (“em”) for each one-minute epoch. The epoch measure is defined as:

$${em}_{m} = \frac{1}{m + S - max ( 0 , m - 5 ) + 1} \text{?} \text{?}$$

$$\text{?} \text{indicates text missing or illegible when filed}$$

Using the epoch measure value, the epoch sampler 630 determines a sleep state for the epoch. The determined sleep state may be a numerical value associated with a sleep state. The numerical value represents an epoch decision, the epoch decision (“edm”) being defined as:

$${ed}_{m} = \{ \begin{matrix}4 & awake & {em}_{m} > {T}_{em - awake} \\ 2 & lightsleep & {T}_{em - deep} < {em}_{m} \leq {T}_{em - awake} \\ 1 & deepsleep & {em}_{m} \leq {T}_{em - deep}\end{matrix}$$

The sleep-decision generator 640 uses a series of epoch decisions to determine a sleep decision for a sleep-decision window, the sleep decision signifying a sleep state over the given sleep-decision window. For example, the sleep-decision generator 640 may use a sleep-decision window of five consecutive epoch values and analyze the epoch values to determine a sleep decision. In some examples the sleep-decision value may be defined as the maximum value of the series of consecutive epochs contained in a sleep-decision window:

$${wd}_{n} = max \text{?} \text{?} , \text{?} , … , \text{?} \text{?}$$

$$\text{?} \text{indicates text missing or illegible when filed}$$

In this example, the maximum value may be used to determine a sleep state.

One or more of the elements, processes, or devices illustrated in FIGS. 4 through 6 may be combined, divided, rearranged, omitted, eliminated, or implemented in another way. Further, the raw-value determiner 420, offset corrector 430, sleep-state determiner 440, data packager/transmitter 450, range determiner 505, boundary determiner 510, incremental-offset corrector 515, offset corrector 520, scaling corrector 525, N-level sampler 610, second sampler 620, minute sampler 630, and sleep-decision generator 640 of FIGS. 4 through 6 may be implemented by hardware, software, firmware, or any combination of hardware, software, or firmware. Thus, these elements may be implemented by one or more circuits, programmable processors, application-specific integrated circuits, programmable logic devices, or field-programmable logic devices, etc. With respect to any claims of this patent that encompass purely software elements, one or more such elements are herein expressly defined to include a tangible non-transitory computer-readable medium such as a memory, DVD, CD, Blu-ray, etc., storing the software or firmware. Further still, the example dynamic calibrator 430 and the example sleep state determiner 440 of FIG. 4 that have been further illustrated in FIGS. 5 and 6, respectively, may include one or more elements, processes, or devices in addition to, or instead of, those illustrated in FIGS. 5 and 6 or may include more than one of any or all of the illustrated elements, processes, and devices.

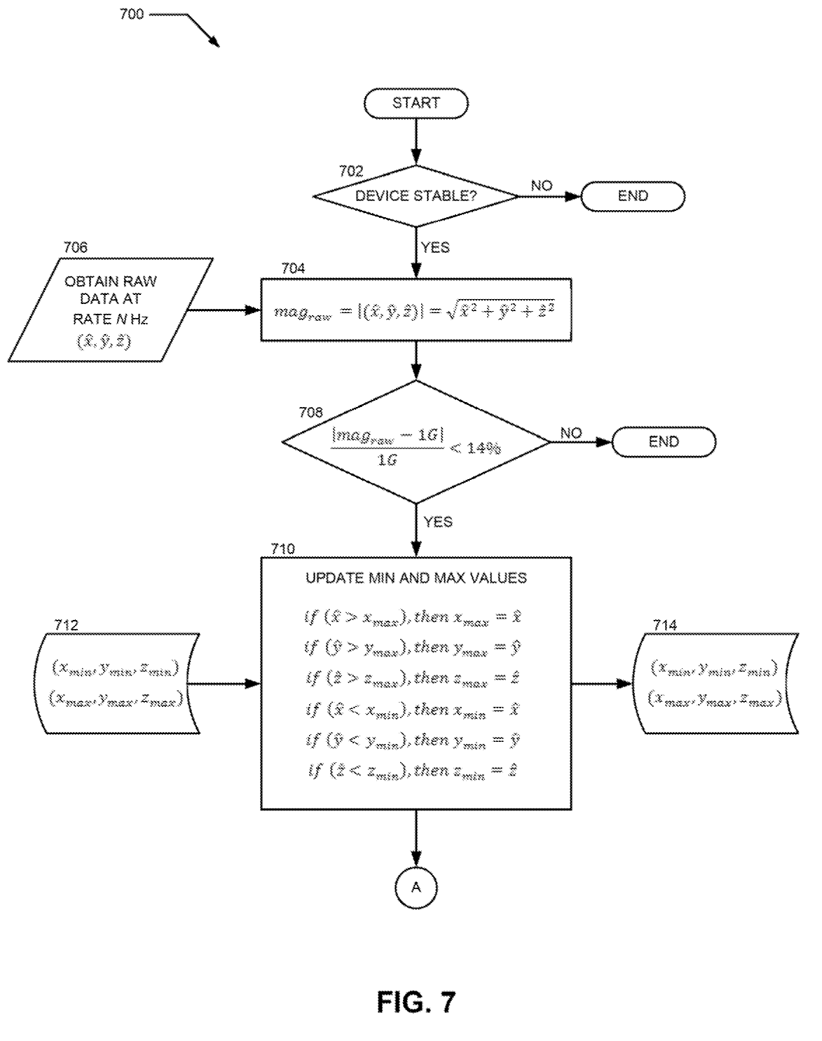

Flowcharts representative of example machine-readable instructions for implementing such elements are shown in FIGS. 7 through 10. In these examples, the machine-readable instructions comprise a program for execution by a processor such as the processor 130 shown in the example user device 110 discussed above in connection with FIG. 1. The program may be embodied in software stored on a tangible computer-readable medium as discussed above. Further, although the example programs are described with reference to the flowcharts illustrated in FIGS. 7 through 10, other methods of implementing disclosed elements may alternatively be used. For example, the order of execution of the steps may be changed, or some of the steps described may be changed, eliminated, or combined.

Continuing, FIG. 7 illustrates a process of obtaining raw data from the accelerometer 115 and updating minimum and maximum values for the vector parameters. At stage 702 of the process 700, stability of the user device 110 is determined. If the device 110 is not in a stable position, then the process 700 ends. However, if the user device 110 is stable, then the 3-D accelerometer data are obtained at a rate of N Hertz (stage 706), and a magnitude of the raw value of 3-D accelerometer data is determined (stage 704). At stage 708, it is determined if the raw magnitude is within a correctable range; if it is not within a correctable range, then the process ends. However, if the raw magnitude is within a correctible range, then the process 700 updates the maximum and minimum values for the 3-D parameters of the accelerometer data by comparing stored prior minimum and maximum values (stage 712) with the current read values. The results of the comparison at stage 710 are stored as the minimum and maximum values for the parameters (stage 714).

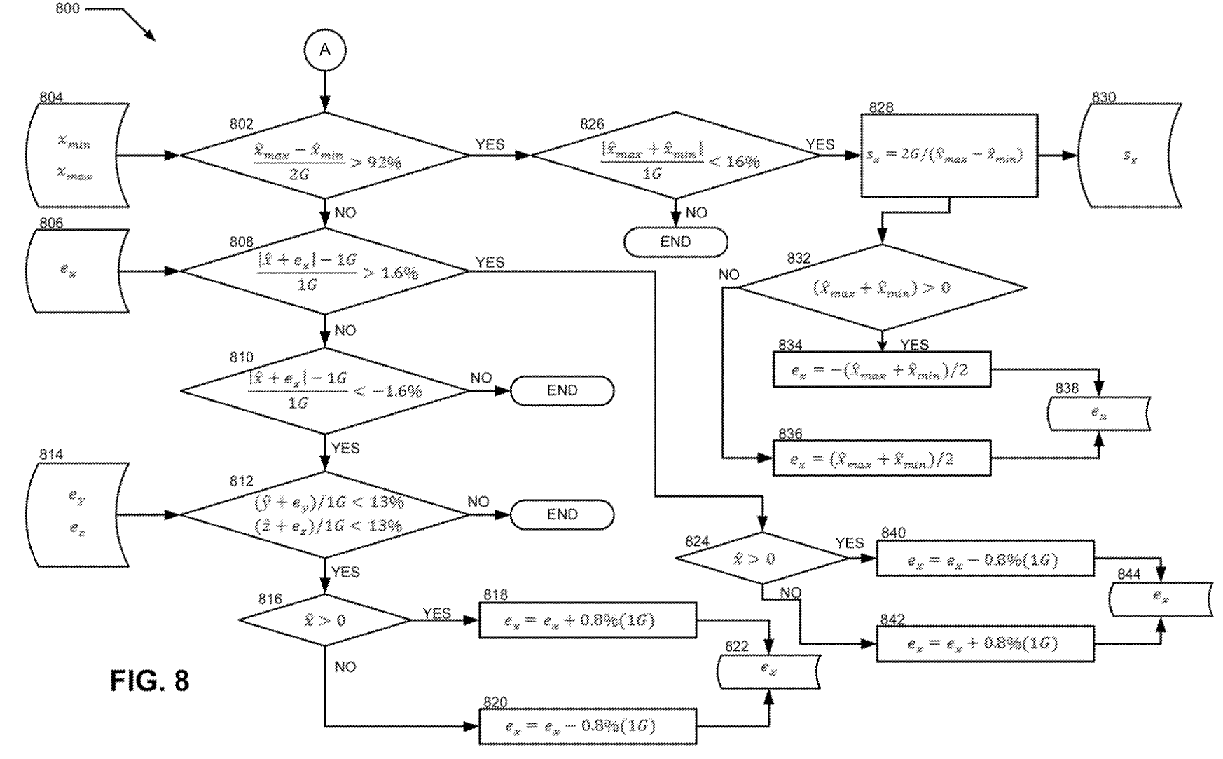

In continuation from FIG. 7, the process 800 of FIG. 8 uses dynamic calibration to determine values for offset correction and scaling correction. For the purposes of illustration, offset and scaling-correction values are shown for the x values of the 3-D acceleration; however, the illustrated process will typically be repeated for each of the y and z axes.

At stage 802, it is determined if the difference between the maximum and minimum values of x meet a minimum separation value. If the minimum and maximum values do not meet the minimum separation value, then the offset may be incrementally adjusted. For example, if the corrected value is too large (stage 808), then the offset may be adjusted as seen in stages 824, 840, and 842. At stage 810, if the magnitude of the corrected value is too small and the corrected values of the other two axes have small magnitudes (stage 812), then the offset may be adjusted as seen in stages 816, 818, and 820. Corrected values for the offset correction are stored to memory.

Returning to stage 802, if the difference between the maximum and minimum values of x meets a minimum separation value, then the process 800 determines if the mean of the maximum and minimum values of x is within a correctable range (stage 826). If the mean of the maximum and minimum values is within a correctable range, then the scaling correction is determined at stage 828 and is stored in memory (stage 830). Further, the offset correction can be determined using the processes of stages 832, 834, and 836. The value determined for the offset correction is then stored to memory (stages 822, 838, 844).

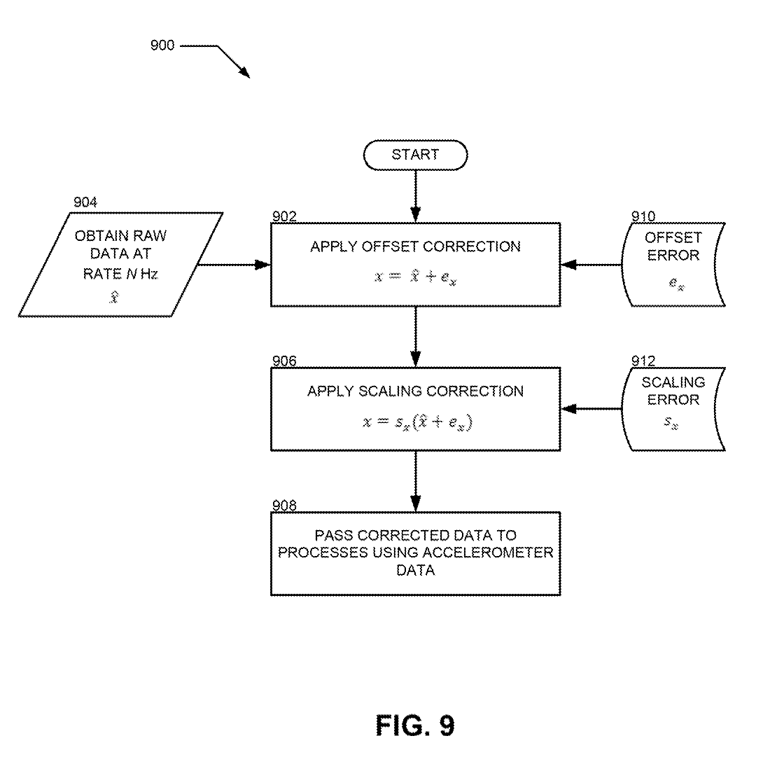

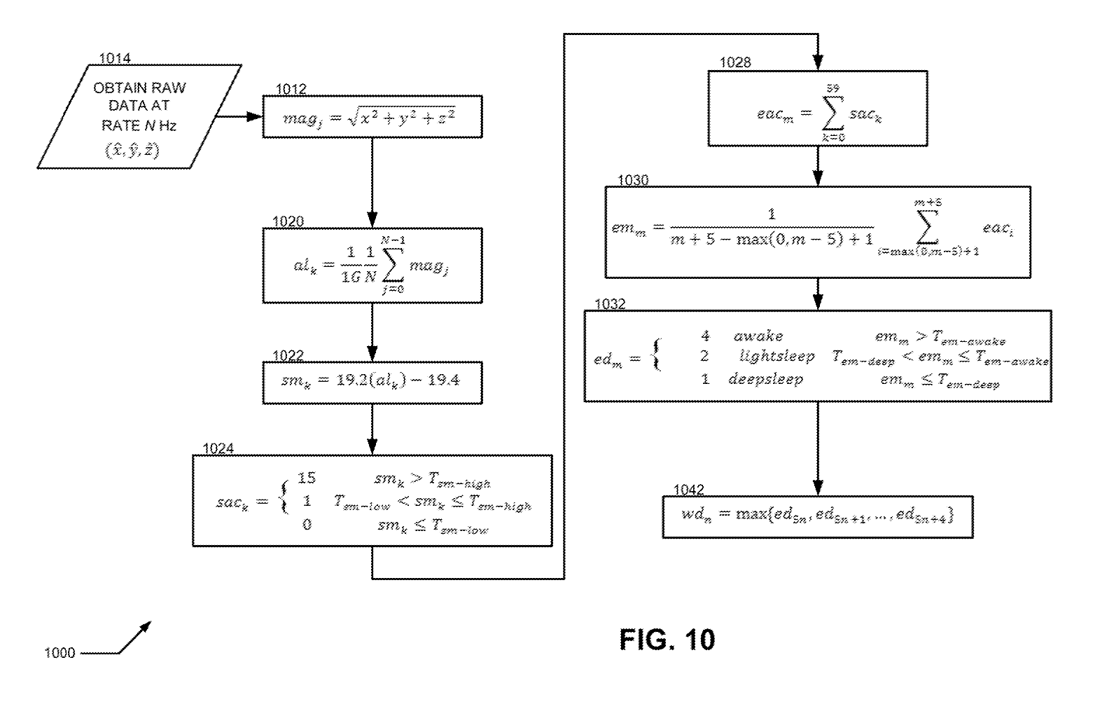

Using the stored values determined by the process 800 of FIG. 8, the process 900 of FIG. 9 applies the offset correction and scaling correction to the obtained raw data at a rate of N Hertz (stage 904). At stage 902, the determined offset correction is applied and is stored in memory (910) by the process 800. At stage 906, the determined scaling correction is applied and stored in memory (912) by the process 800. At stage 908, the process 900 passes the corrected accelerometer data to processes that use accelerometer data (i.e., the sleep-analysis process of FIG. 10).

FIG. 10 is a flowchart of a sample process 1000 for determining a sleep state from a series of samples of accelerometer data. At stage 1014, accelerometer data are received at a rate of N Hertz. A magnitude value is determined from each sample (stage 1012).

Using the magnitude values determined at stage 1012, the process 1000 determines an average normalized activity level over N samples (stage 1020). A sample measure is determined using the average normalized activity level (stage 1022). Using the sample measure value and predetermined high and low thresholds for the sample measure, a sample activity count is determined (stage 1024).

Using a series of k sample activity counts, an epoch activity count is determined for a 60-sample epoch (stage 1028). An epoch measure for each epoch is then determined using the epoch activity count values (stage 1030). Using the epoch measure value, an epoch decision value is determined (stage 1032). A sleep-decision value for a sleep-decision window can then be determined by receiving a series of epochs (i.e., five consecutive epochs) and determining the maximum value from the series of epochs (stage 1042). As noted above, the sleep-decision value is associated with a sleep state: For example, the sleep-decision value may be compared against reference values to determine if the user is experiencing light sleep, deep sleep, or is awake.

In view of the many possible embodiments to which the principles of the present discussion may be applied, it should be recognized that the embodiments described herein with respect to the drawing figures are meant to be illustrative only and should not be taken as limiting the scope of the claims. Therefore, the techniques as described herein contemplate all such embodiments as may come within the scope of the following claims and equivalents thereof.

We claim:

1. A method for determining a sleep state of a user, the method comprising:
receiving motion-based data samples from an accelerometer associated with a user device worn by the user; and
calculating, with a processor, a sleep-state value for a plurality of the motion-based data samples, wherein the sleep-state value is determined by:
determining one or more average normalized activity levels, each average normalized activity level determined from a series of motion-based data samples received from the accelerometer;
determining an epoch decision value, wherein the epoch decision value is determined based on one or more consecutive normalized activity levels;
determining a sleep-decision window value, wherein the sleep-decision window comprises a series of one or more consecutive epoch decision values;
determining a sleep-state value, wherein the sleep-state value is determined from the epoch decision values of the series of one or more consecutive epoch decision values from which the sleep-decision window is determined; and
comparing the sleep-state value with a plurality of reference sleep-state values to determine the sleep state of the user.

2. The method of claim 1 wherein the sleep-state value is determined by choosing a greatest epoch decision value from the epoch decision values from which the sleep-decision window is determined.

3. The method of claim 1 further comprising calibrating the accelerometer via dynamic calibration.

4. The method of claim 1 wherein the sleep state is one of light sleep, deep sleep, and awake.

5. The method of claim 1 wherein the user device is a wrist watch.

6. The method of claim 1 wherein each motion-based data sample reflects a magnitude of an acceleration associated with the accelerometer.

7. The method of claim 1 wherein the average normalized activity levels are determined from N motion-based data samples over a period of one second.

8. The method of claim 7 wherein the epoch decision value is determined using a series of one or more consecutive sample-activity counts, wherein each sample-activity count is determined by processing one of the normalized activity counts of the epoch decision value using predetermined high and low threshold values.

9. The method of claim 8 wherein the epoch decision value is determined by summing a series of one or more sample activity counts and applying predetermined thresholds to determine an activity count associated with a sleep state.

10. The method of claim 9 wherein the series of one or more sample activity counts is a series of 60 sample activity counts.

11. The method of claim 10 wherein the sleep-decision window comprises five epoch decision values.

12. A method for calibrating motion-based data samples received from an accelerometer, the method comprising:
receiving a motion-based data sample from the accelerometer, the data sample associated with an axis, wherein the axis is an axis of a Cartesian coordinate system;
determining, using previously stored motion-based samples associated with the axis and the received motion-based data sample, if the difference between a minimum value and a maximum value for the coordinate is within an acceptable range;
if the difference between the minimum value and the maximum value for the coordinate is not within the acceptable range, then incrementally adjusting a first offset correction until the difference between the minimum value and the maximum value is within the acceptable range;
if the difference between the minimum value and the maximum value for the coordinate is within the acceptable range, then determining if a mean value of the minimum value and the maximum value for the coordinate is within a correctable range; and
if the mean value of the minimum value and the maximum value for the coordinate is within the correctable range, then determining:
a scaling correction; and
a second offset correction, based on the first offset correction value; and
calibrating the motion-based data samples based on the scaling correction and the second offset correction.

13. The method of claim 12 wherein each motion based data sample is an acceleration vector.

14. The method of claim 13 wherein the acceleration vector is a three-dimensional vector.

15. The method of claim 12 further comprising using a stability threshold function to turn off the accelerometer when it is stable.

16. A user device for determining a sleep state of a user, the user device comprising:
an accelerometer;
a raw-value determiner for receiving motion-based data samples from the accelerometer and for determining a plurality of raw acceleration values;
a calibrator for detecting and for correcting calibration errors in the raw acceleration values to produce calibrated acceleration values; and
a sleep-state determiner configured to determine one or more average normalized activity levels from the calibrated acceleration values, to determine an epoch decision value based on one or more consecutive normalized activity levels, to identify a sleep-decision window encompassing a series of one or more consecutive epoch decision values, to determine a sleep-state value from the one or more consecutive epoch decision values of the sleep-decision window, and to compare the sleep-state value to a plurality of reference sleep-state values to determine the sleep state of the user.

17. The user device of claim 16 wherein the sleep-state determiner is further configured to determine the sleep-state value by choosing a greatest epoch decision value from the epoch decision values of the sleep-decision window.

18. The user device of claim 16 wherein the determined sleep state is one of light sleep, deep sleep, and awake.

19. The user device of claim 16 wherein the sleep-decision window encompasses five epoch decision values.

20. The user device of claim 16 wherein the device is a wrist watch.
